(19) United States
(12) Patent Application Publication
(10) Pub. No.: US 2013/0171724 A1
(43) Pub. Date: Jul. 4, 2013

(54) CHEMICAL REACTION VESSELS

(71) Applicant: Abbott Molecular Inc., Des Plaines, IL (US)

(72) Inventors: John M. Clemens, Wadsworth, IL (US); Curtis J. Pepe, McHenry, IL (US); Herbert A. Marble, Naperville, IL (US); Donald Joseph Home, Los Gatos, CA (US)

(73) Assignee: ABBOTT MOLECULAR INC., Des Plaines, IL (US)

(21) Appl. No.: 13/729,930

(22) Filed: Dec. 28, 2012

Related U.S. Application Data
(60) Provisional application No. 61/741,762, filed on Dec. 30, 2011.

(51) Int. Cl. C12M 1/32

(52) U.S. Cl. CPC C12M 23/12
USPC 435/289.1; 29/412

(57) ABSTRACT

Chemical reaction vessels are disclosed herein. An example method includes forming a reaction chamber between two pliable sheets and dividing the reaction chamber into a plurality of chambers. The reaction chamber is to have a substantially planar base, and the reaction chamber is to hold a fluid for a chemical reaction.

### RELATED APPLICATION

This patent claims priority to U.S. Provisional Patent Application Ser. No. 61/741,762, entitled “Chemical Reaction Vessels,” which was filed on Dec. 30, 2011, and which is incorporated herein by reference in its entirety.

### FIELD OF THE DISCLOSURE

This disclosure relates generally to chemical reactions and, more particularly, to chemical reaction vessels.

### BACKGROUND

Real-time polymerase chain reaction (PCR) includes a chemical reaction that may be used to amplify, detect and quantify an initial concentration of a target DNA or RNA molecule in a biological specimen. To perform real-time PCR, a mixture of a heat-stable polymerase, primers, deoxynucleotides, salts, additives and a buffer may be repeatedly heated and cooled. The mixture is often dispensed into tubes and then placed inside a thermal cycler, which thermally cycles (i.e., heats and cools) the mixture in the tubes. The chemical reaction may be monitored by determining an emission of fluorescence by, for example, using intercalating dyes and/or fluorescent reporter probes. To quantify the concentration of the target DNA or RNA molecules, the mixture may be cycled between about 30 and about 45 times.

Real-time PCR may be used for molecular diagnostic applications such as, for example, viral load monitoring, infectious disease screening and diagnosis (e.g., HPV genotyping, CT/NG, TB, MRSA, hospital acquired infections, etc.), and MRNA expression analysis. Digital PCR (dPCR) may quantify and enumerate DNA and RNA molecules by, for example, end-point limiting dilution analysis (i.e., Poisson analysis) or clonally amplifying physically discrete populations of nucleic acids. Digital PCR may detect and enumerate single molecules. In Digital PCR, the mixture may be homogeneous. The mixture may be distributed into a plurality of chambers to stochastically confine zero, one or more target DNA molecules. Digital PCR may be used to detect genomic aberrations in cancer such as, for example, copy number variation; perform single nucleotide polymorphism analysis; and detect somatic alleles. Digital PCR may also be used for sensitive pathogen identification/discrimination, antibiotic resistance profiling, high-multiplex genotyping and non-invasive circulating fetal DNA analysis from maternal blood.

### BRIEF DESCRIPTION OF THE DRAWINGS

FIG. 1A depicts a top view of an example apparatus disclosed herein.

FIG. 1B depicts a cross-sectional, side view of the example apparatus of FIG. 1A.

FIG. 2 illustrates example pliable sheets that may be used to implement example methods and apparatus disclosed herein.

FIG. 3 illustrates an example reaction chamber and an example mixing chamber disclosed herein.

FIG. 4 illustrates the example mixing chamber of FIG. 3 holding a fluid.

FIG. 5 illustrates the fluid of FIG. 4 flowing into the example reaction chamber.

FIG. 6 illustrates the example reaction chamber holding the fluid.

FIG. 7 illustrates the example reaction chamber divided into a plurality of chambers.

FIG. 8 illustrates the example reaction chamber including a brace.

FIG. 9 illustrates the example reaction chamber separated from the pliable sheets.

FIGS. 10-11 illustrate a flow chart representative of an example method disclosed herein.

### DETAILED DESCRIPTION

While the following example apparatus and methods disclosed herein are described in conjunction with polymerase chain reaction (PCR) analysis, the example apparatus and methods may also be used for performing any other chemical reactions or processes including a chemical reaction. The example methods and apparatus disclosed herein may be used for performing polymerase chain reaction (PCR). For example, real-time PCR may be used to amplify, detect and quantify an initial concentration of a target DNA or RNA molecule in a biological specimen (e.g., whole blood, lymphatic fluid, serum, plasma, buccal, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.) or washes (e.g., oral, nasopharangeal, bronchial, bronchialalveolar, optic, rectal, intestinal, vaginal, epidermal, etc.) and/or other specimens). A quantity of DNA may be expressed as a number of molecules or a relative amount (i.e., proportion) of molecules normalized to a DNA input, calibrator, and/or endogenous reference gene. To perform real-time PCR, a mixture of a heat-stable polymerase, primers, deoxynucleotides, salts, additives and a buffer may be placed in reaction chambers and then repeatedly heated and cooled inside a real-time thermal cycler. A fluorescence emission may be determined using, for example intercalating agents (e.g., SYBR Green™), hydrolysis probes (e.g., TaqMan™), conformational probes (e.g., molecular beacons), or quantum dot probes. To quantify the initial concentration of the target DNA or RNA molecules, the fluorescence emission may be determined while the mixture is heated and cooled.

Digital PCR (dPCR) may quantify and enumerate DNA and RNA molecules by, for example, end-point limiting dilution analysis (i.e., Poisson analysis) or clonally amplifying physically discrete populations of nucleic acids. Digital PCR may detect and enumerate single molecules. In Digital PCR, the mixture may be homogeneous. In some examples, the mixture may be distributed into a plurality of chambers separated by structural or emulsive barriers to stochastically confine zero, one or more target DNA molecules. In other examples, holding the mixture in a single, thin (e.g., a thickness or height of about 50 microns to about 200 microns) chamber may provide a spatial confinement of DNA targets without separating the DNA targets using structural or emulsive barriers. In such examples, focal fluorescent signals emitted by isolated and spatially confined amplicon clusters may be enumerated, and initial copies of the DNA targets may be quantified by, for example, counting the DNA targets. A fluorescence emission may be determined using, for example intercalating dyes and/or fluorescent reporter probes.

The example apparatus and methods disclosed herein may enable the mixture to be heated and cooled during a real-time or digital PCR process at a rate of about twenty degrees Celsius per second. An example method disclosed herein includes forming a reaction chamber between two pliable sheets. In some examples, the reaction chamber is to hold a fluid for a chemical reaction, such as, for example, a PCR reaction. The reaction chamber may have a substantially planar base. In some examples, the sheets are dispensed from one or more rolls of pliable material. One or more of the sheets may be translucent or substantially transparent. In some examples, the reaction chamber is divided into a plurality of reaction chambers. In some examples, the reaction chamber is sealed. A mixing chamber may be formed adjacent the reaction chamber and fluidly coupled to the reaction chamber. A removable seal may be formed between the mixing chamber and the reaction chamber. In some examples, the reaction chamber is braced and/or separated from the sheets. In some examples, the reaction chamber has a thickness of about 50 microns to about 200 microns, and the base of the reaction chamber may include scores.

An example apparatus disclosed herein includes a first pliable sheet coupled to a second pliable sheet to define a reaction chamber between the sheets. One or more of the sheets may be translucent or substantially transparent. In some examples, the reaction chamber has a substantially planar base. The example apparatus may include a brace to impart rigidity to the reaction chamber. In some examples, the reaction chamber is to hold a fluid for a chemical reaction such as, for example, a PCR reaction. The first pliable sheet and the second pliable sheet may further define a mixing chamber fluidly coupled to the reaction chamber. Some such examples may include a removable seal between the mixing chamber and the reaction chamber. In some examples, the base of the reaction chamber includes scores. In some examples, the reaction chamber includes a plurality of chambers. The example apparatus may include a plurality of walls disposed in the reaction chamber to divide the reaction chamber into the plurality of chambers. In some examples, the walls extend from the first pliable sheet to the second pliable sheet. The sheets may be dispensed from one or more rolls of pliable material. In some examples, the reaction chamber has a thickness between about 50 microns and about 200 microns.

Another example method disclosed herein includes coupling a first pliable sheet to a second pliable sheet to define a first chamber and a second chamber fluidly coupled to the first chamber. One or more of the first and second pliable sheets may be translucent or substantially transparent. The first chamber has a thickness between about 50 microns and about 200 microns. In some examples, the method further includes providing a brace to impart rigidity to the first chamber. In some examples, the first chamber is divided into a plurality of chambers. In some examples, a base of the first chamber is substantially planar. The base may include scores. The first chamber may hold a fluid for PCR amplification. In some examples, a removable seal is formed between the first chamber and the second chamber. In some examples, the first chamber is sealed.

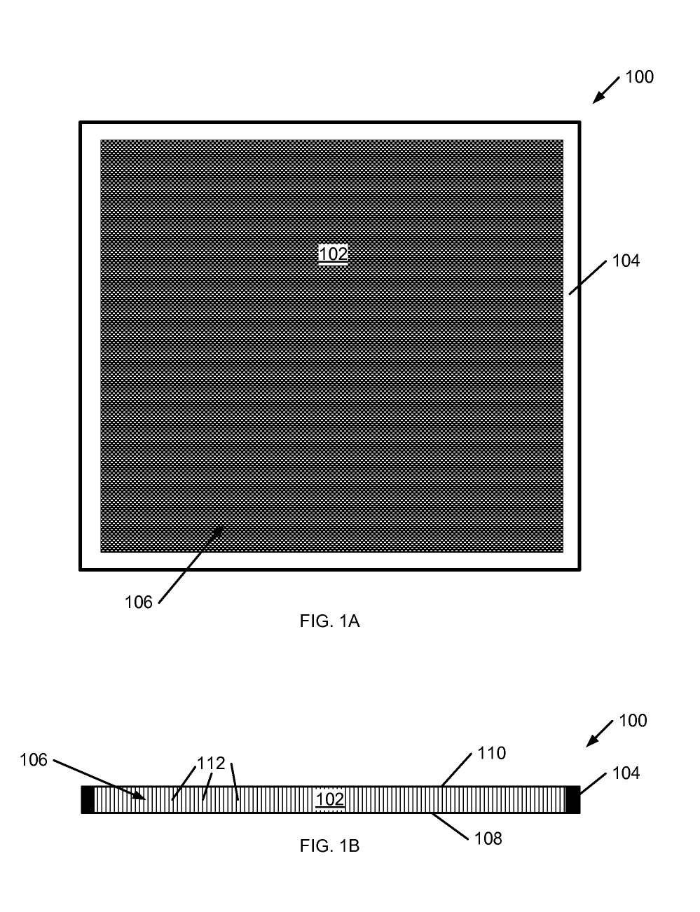

FIGS. 1A and 1B illustrate an example apparatus 100 disclosed herein. The example apparatus 100 includes a reaction chamber 102 and a brace 104. In some examples, the brace 104 is coupled to the reaction chamber 102 to impart rigidity to the reaction chamber 102. In the illustrated example, the reaction chamber 102 includes a plurality of chambers 106. In some examples, the reaction chamber 102 does not include a plurality of chambers. The example reaction chamber of FIGS. 1A and 1B includes a substantially planar base 108. In some examples, a top 110 of the example reaction chamber 102 is also substantially planar. A thickness of the reaction chamber 102 is about 50 microns to about 200 microns. In the illustrated example, walls 112 of the plurality of chambers 106 extend from the base 108 to the top 110 of the reaction chamber 102. As described in greater detail below, the reaction chamber 102 is defined between two pliable sheets, which may form the base 108, the top 110 and/or the walls 112.

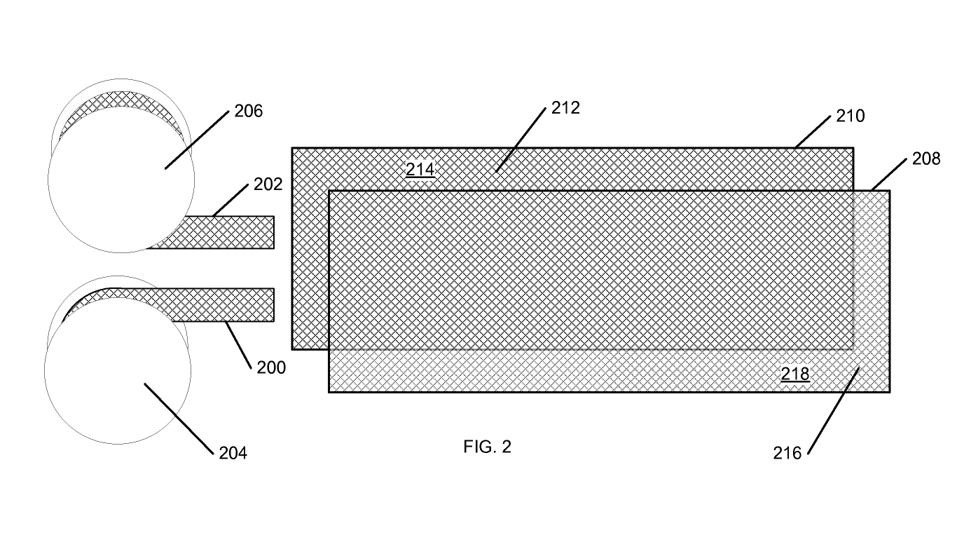

FIG. 2 depicts example first and second pliable materials 200 and 202 that may be used to implement example methods and apparatus disclosed herein. The materials 200 and 202 may be the same material or different materials to suit the needs of a particular application. For example, the first and second pliable materials 200 and 202 may be plastic and/or any other suitable material. In the illustrated example, the first material 200 and the second material 202 are dispensed from rolls 204 and 206 to form first and second respective sheets 208 and 210. In some examples, the first sheet 208 and/or the second sheet 210 are translucent or substantially transparent. The second sheet 210 includes scores 212 on a face 214 facing the first sheet 208, and the first sheet 208 includes scores 216 on a face 218 facing the second sheet 210. In the illustrated example, the scores 212 and 216 intersect at substantially right angles. In some examples, the scores 212 and 216 intersect at angles less than ninety degrees. The scores 212 and 216 may intersect at a variety of angles. In some examples, the scores 216 on the face 218 of the first sheet 208 are parallel, and/or the scores 212 on the face 214 of the second sheet 210 are parallel. In such examples, the scores 216 on the face 218 of the first sheet 208 may be perpendicular and/or any other suitable angle relative to the scores 212 on the face 214 of the second sheet 210.

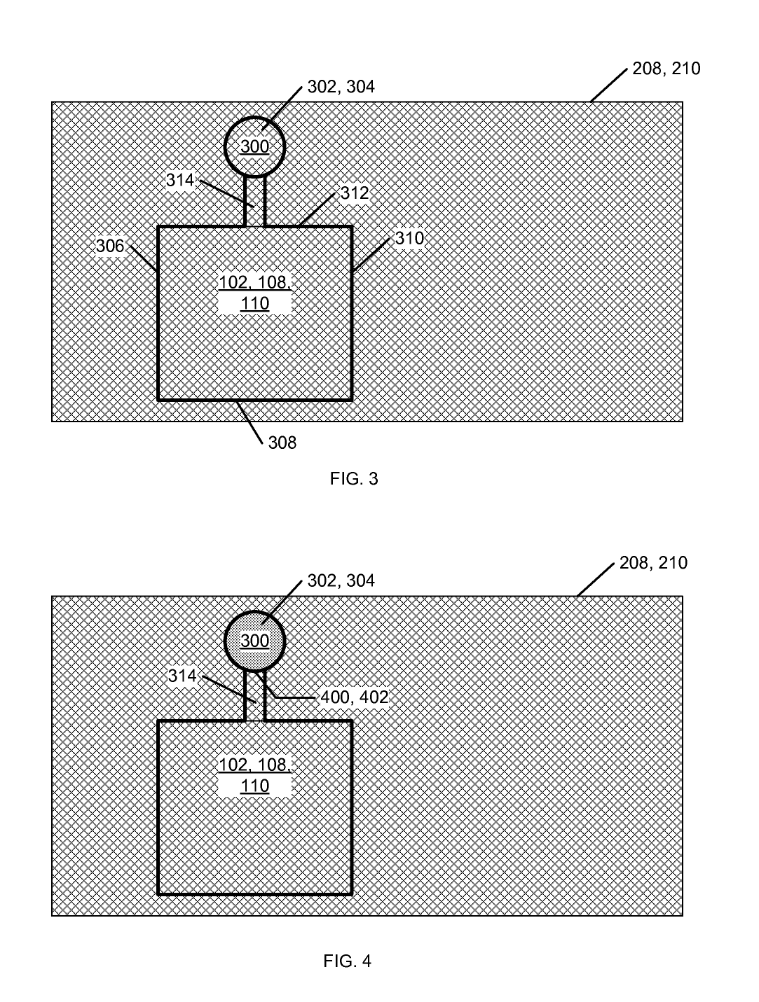

FIG. 3 illustrates the example reaction chamber 102 and an example mixing chamber 300. As the sheets 208 and 210 are dispensed, the first sheet 208 is coupled to the second sheet 210 to form the reaction chamber 102 between the first sheet 208 and the second sheet 210. In the illustrated example, a mixing chamber 300 is also formed between the first sheet 208 and the second sheet 210. For example, the first sheet 208 is coupled to the second sheet 210 via pressing the sheets 208 and 210 and/or welding (e.g., ultrasonically welding) the sheets 208 and 210 together. In some examples, the reaction chamber 102 and the mixing chamber 300 are formed using a die. In the illustrated example, the second sheet 210 defines the base 108 of the reaction chamber 102 and a base 302 of the mixing chamber 300. The first sheet 208 defines the top 110 of the reaction chamber 102 and a top 304 of the mixing chamber 300. In the illustrated example, the base 108 of the reaction chamber 102 is rectangular, and the base 302 of the mixing chamber 300 is circular. However, the above-noted shapes of the bases 108 and 302 of the reaction chamber 102 and the mixing chamber 300 are merely examples and, thus, other shapes may be used without departing from the scope of this disclosure. In some examples, sides 306, 308, 310 and 312 of the reaction chamber 102 are between about 1 cm and about 3 cm in length. A thickness or height of the reaction chamber 102 is about 50 microns to about 200 microns. In some examples, the reaction chamber 102 is to hold a volume of about 25 ml to about 100 ml of fluid.

The reaction chamber 102 is fluidly coupled to the mixing chamber 300. In the illustrated example, the reaction chamber 102 is fluidly coupled to the mixing chamber 300 via a channel 314 formed between the reaction chamber 102 and the mixing chamber 300. As discussed in greater detail below, some examples include a removable fluid seal between the reaction chamber 102 and the mixing chamber 300.

FIG. 4 illustrates the example mixing chamber 300 holding a fluid. In some examples, after the reaction chamber 102 and the mixing chamber 300 are formed, the fluid is discharged into the mixing chamber 300. The fluid may include a heat-stable polymerase, primers, deoxynucleotides, salts, additives, one or more surface-passivating agents and a buffer for polymerase chain reaction (PCR). The illustrated example includes a removable seal 400 to prevent the fluid from flowing into the reaction chamber 102. In the illustrated example, the removable seal 400 is a frangible edge 402 of the mixing chamber 300. The seal 400 may be removed by, for example, separating or fracturing the seal 400 via twisting, stretching, pulling, heating, and/or any other suitable method of removing the seal 400.

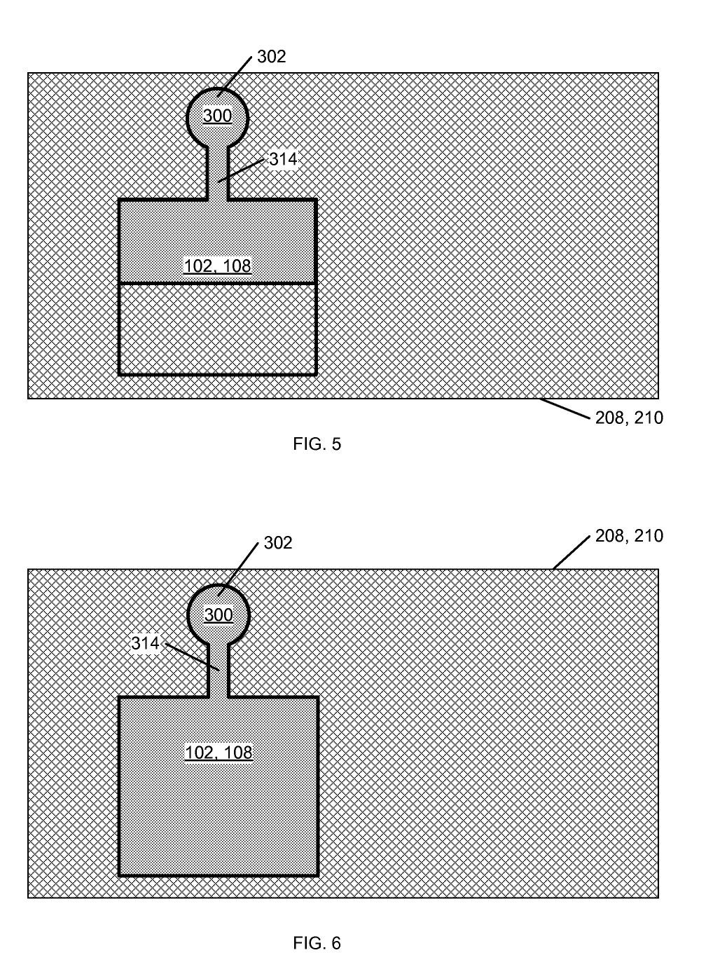

FIGS. 5 and 6 illustrate the fluid flowing from the example mixing chamber 300 to the example reaction chamber 102. In some examples, the base 302 of the mixing chamber 300 is disposed closer to the face 218 of the first sheet 208 facing the second sheet 210 than the base 108 of the reaction chamber 102. In such examples, the channel 314 may slope toward the base 108 of the reaction chamber 102. In the illustrated examples, the seal 400 is removed, and the fluid flows from the mixing chamber 300 to the reaction chamber 102. In FIG. 6, the reaction chamber 102 holds a volume of about 25 ml to about 100 ml of fluid. The above-noted volume is merely an example and, thus, other volumes may be used without departing from the scope of this disclosure.

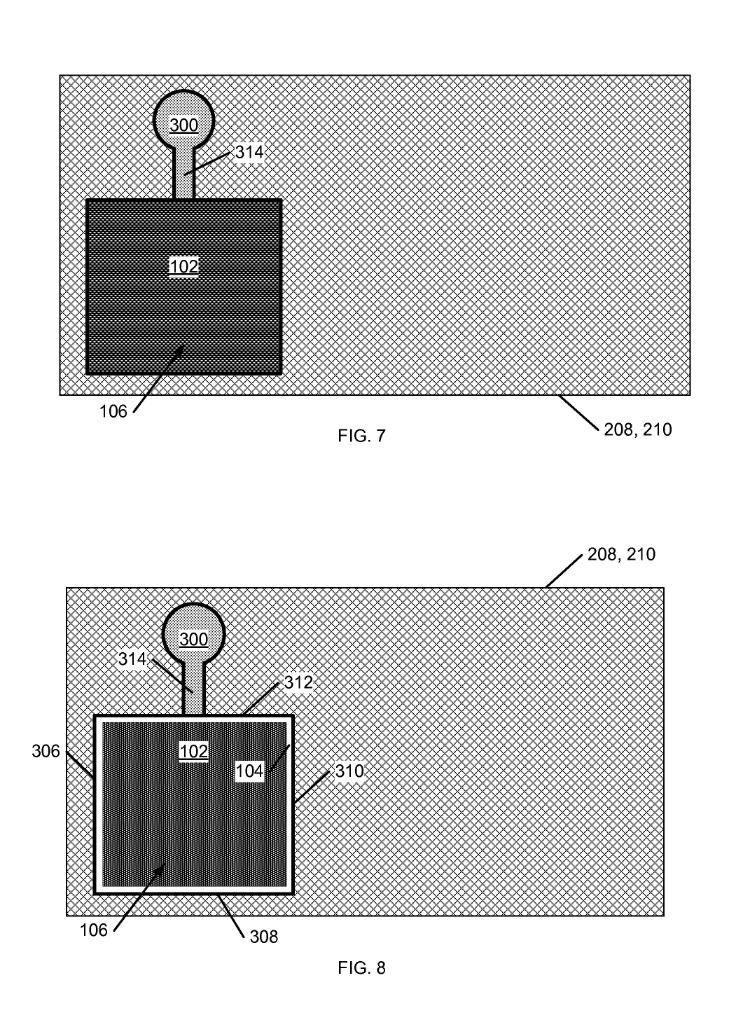

FIG. 7 illustrates the example reaction chamber 102 including the plurality of chambers 106. In some examples, the reaction chamber 102 is divided into the plurality of chambers 106. For example, the second sheet 210 and the first sheet 208 may be pressed and/or welded (e.g., ultrasonically welded) together to form the plurality of chambers 106. In some examples, the second sheet 210 and the first sheet 208 are pressed and/or welded to enable the second sheet 210 and/or the first sheet 208 to form the walls 112 to divide the reaction chamber 102 into the plurality of chambers 106. In the illustrated example, the reaction chamber 102 includes 1024 chambers, and each of the chambers 106 is to hold a volume of about 50 nl of fluid. For example, the chambers 106 may have dimensions of 1 mm×1 mm×0.05 mm, 0.71 mm×0.71 mm×0.1 mm, or 0.5 mm×0.5 mm×0.2 mm. The above noted dimensions are merely examples and, thus, other dimensions may be used without departing from the scope of this disclosure. In other examples, the reaction chamber 102 is not divided into the plurality of chambers 106.

FIG. 8 illustrates the example brace 104 coupled to the reaction chamber 102. In some examples, the brace 104 is coupled to the reaction chamber 102 via the first sheet 208. In other examples, the brace 104 is coupled to the reaction chamber 102 via the second sheet 210. In other examples, the brace 104 is disposed between the first sheet 208 and the second sheet 210 and may be coupled to the first sheet 208 and/or the second sheet 210. The example brace 104 imparts rigidity to the reaction chamber 102. In the illustrated example, the brace 104 is framed around the sides 306, 308, 310 and 312 of the reaction chamber 102. However, the above-noted shape of the brace 104 is merely an example and, thus, other shapes may be used without departing from the scope of this disclosure. The brace 104 may be bonded and/or welded to one or more of the sheets 208 and 210. In the illustrated example, the brace 104 seals the reaction chamber 102 (e.g., the brace 104 substantially forms a fluid seal between the first sheet 208 and the second sheet 210 that substantially extends around the sides 306, 308, 310 and 312). In some examples, the reaction chamber 102 is sealed by pressing, welding, and/or heating the sheets 208 and 210 and/or any other suitable method of sealing the reaction chamber 102.

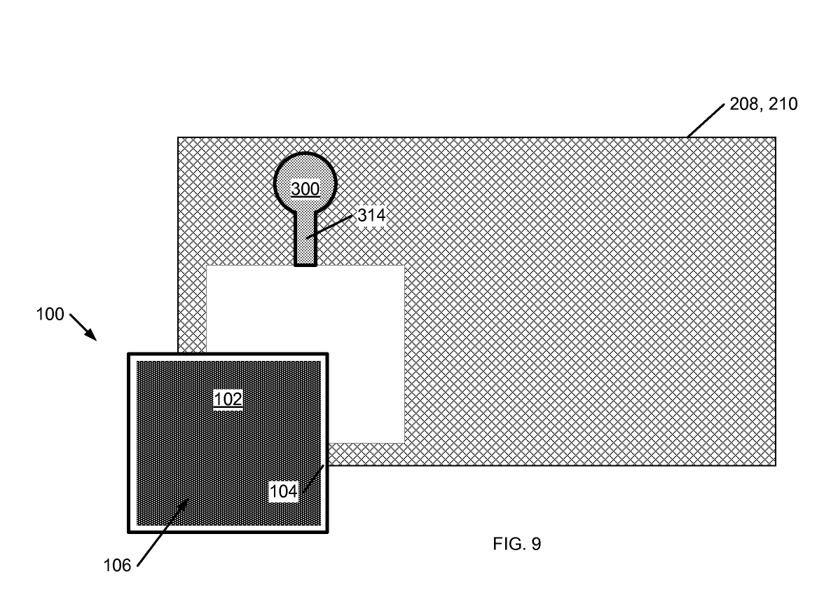

FIG. 9 illustrates the example apparatus 100 separated from the sheets 208 and 210. For example, the reaction chamber 102 may be cut and/or stamped out of the sheets 208 and 210. The reaction chamber 102 may then be used to perform PCR. In some such examples, the reaction chamber 102 may be placed in a thermal cycler (not shown) and cycled at a rate of about twenty degrees Celsius per second. For example, air may be directed to contact the first sheet 208 and/or the second sheet 210 to heat and/or cool the reaction chamber 102 and, thus, the fluid in the reaction chamber 102. In some examples, the brace 104 enables the base 108 of the reaction chamber 102 to be substantially planar while the example apparatus 100 is heated and cooled during the PCR process. As disclosed above, the first sheet 208 and/or the second sheet 210 may be translucent or substantially transparent. As a result, optical viewing, reading and/or imaging of one or more chemical reactions (e.g., one or more PCR chemical reactions) within the reaction chamber 102 (e.g., within one or more of the plurality of the chambers 106) may be performed discretely and/or as an array via the base 108 (e.g., the first sheet 208) and/or the top 110 (e.g., the second sheet 210) of the reaction chamber 102.

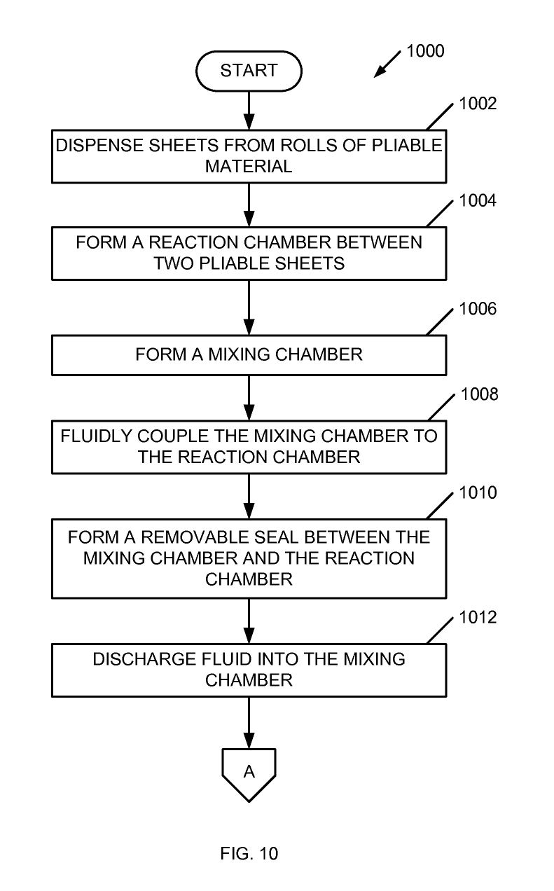

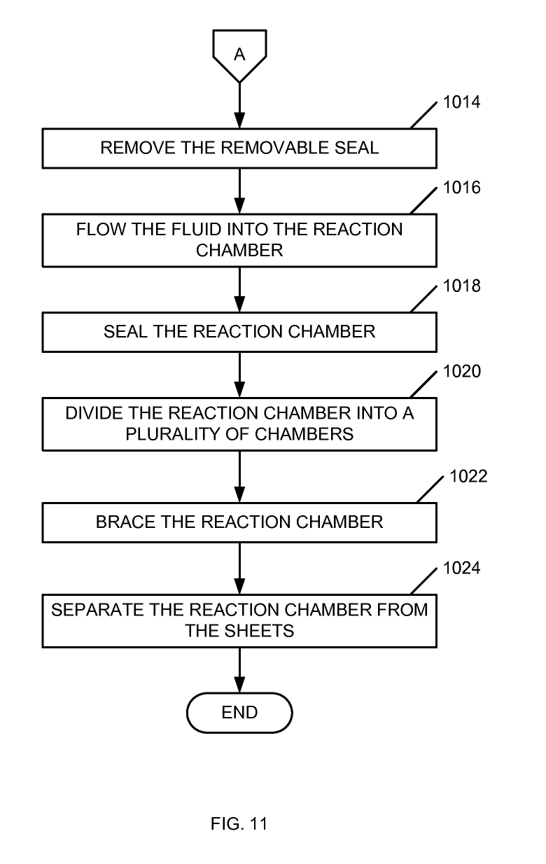

FIGS. 10-11 depict a flow diagram representative of an example method disclosed herein. Although the example method of FIGS. 10-11 is described with reference to the flow diagram of FIGS. 10-11, other methods of implementing the method of FIGS. 10-11 may be employed. For example, the order of execution of the blocks may be changed, and/or some of the blocks described may be changed, eliminated, sub-divided, or combined. Additionally, one or more of the operations depicted in FIGS. 10-11 may be performed sequentially and/or in parallel by, for example, separate persons and/or devices.

With reference to FIGS. 1-9, the example method 1000 of FIGS. 10-11 begins by dispensing the sheets 208 and 210 from one or more rolls 204 and 206 of pliable material (block 1002). At block 1004, the reaction chamber 102 is formed between the two pliable sheets 208 and 210. For example, the first sheet 208 is coupled to the second sheet 210 via pressing the sheets 208 and 210 and/or welding (e.g., ultrasonically welding) the sheets 208 and 210 together. The base 108 of the reaction chamber 102 is substantially planar and may include scores 212. The top 110 of the reaction chamber 102 may also be substantially planar and include scores 216. For example, both of the faces 214 and 218 of the sheets 208 and 210 defining the reaction chamber 102 may include scores 212 and 216. The first sheet 208 and/or the second sheet 210 may be translucent or substantially transparent. A thickness of the reaction chamber 102 is about 50 microns to about 200 microns. In some examples, the reaction chamber 102 is to hold a volume of about 25 ml to about 100 ml of fluid.

At block 1006, the mixing chamber 300 is formed. At block 1008, the mixing chamber 300 is fluidly coupled to the reaction chamber 102. In some examples, the mixing chamber 300 is fluidly coupled to the reaction chamber 102 via the channel 314 between the reaction chamber 102 and the mixing chamber 300. At block 1010, a removable seal 400 is formed between the mixing chamber 300 and the reaction chamber 102. The removable seal 400 may be the frangible edge 402 of the mixing chamber 300. The mixing chamber 300, the channel 314, and the removable seal 400 may be formed when the sheets 208 and 210 are pressed and/or welded together to form the reaction chamber 102. At block 1012, a fluid is discharged into the mixing chamber 300. The fluid may include a heat-stable polymerase, primers, deoxynucleotides, salts, additives, one or more surface-passivating agents and a buffer for polymerase chain reaction (PCR). At block 1014, the removable seal 400 is removed. For example, the seal 400 is removed via separating and/or fracturing the seal 400. The fluid is then flowed into the reaction chamber 102 (block 1016). A volume of about 25 ml to about 100 ml of fluid may flow into the reaction chamber 102.

At block 1018, the reaction chamber 102 is sealed. For example, the sheets 208 and 210 are pressed and/or welded to seal the reaction chamber 102. At block 1020, the reaction chamber 102 is divided into a plurality of chambers 106. For example, the second sheet 210 and the first sheet 208 may be pressed and/or welded to form the plurality of chambers 106 such as, for example, 1024 of the chambers 106. In some examples, the sheets 208 and 210 are pressed and/or welded to form the walls 112 to divide the reaction chamber 102 into the plurality of chambers 106. At block 1022, the reaction chamber 102 is braced. For example, the brace 104 may be disposed around the sides 306, 308, 310 and 312 of the reaction chamber 102. In some examples, the brace 104 is bonded and/or welded to the sheets 208 and 210. The brace 104 may seal the reaction chamber 102. At block 1024, the reaction chamber 102 is separated from the sheets 208 and 210. For example, the reaction chamber 102 may be cut and/or stamped out of the sheets 208 and 210. The reaction chamber 102 may then be used to perform a PCR process.

In such examples, the reaction chamber 102 may be placed in a thermal cycler and heated and cooled using, for example, air at a rate of about twenty degrees Celsius per second.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

The Abstract at the end of this disclosure is provided to comply with 37 C.F.R. §1.72(b) to allow the reader to quickly ascertain the nature of the technical disclosure. It is submitted with the understanding that it will not be used to interpret or limit the scope or meaning of the claims.

What is claimed is:

1. A method, comprising:
forming a reaction chamber between two pliable sheets, the reaction chamber to have a substantially planar base, wherein the reaction chamber is to hold a fluid for a chemical reaction; and
dividing the reaction chamber into a plurality of chambers.

2. The method of claim 1, further comprising dispensing the sheets from one or more rolls of pliable material.

3. The method of claim 1, further comprising bracing the reaction chamber.

4. The method of claim 1, further comprising sealing the reaction chamber.

5. The method of claim 1, further comprising:
forming a mixing chamber adjacent the reaction chamber; and
fluidly coupling the mixing chamber to the reaction chamber.

6. The method of claim 5, further comprising forming a removable fluid seal between the mixing chamber and the reaction chamber.

7. The method of claim 1, further comprising separating the reaction chamber from the sheets.

8. The method of claim 1, wherein the reaction chamber has a thickness of about 50 microns to about 200 microns.

9. The method of claim 1, wherein the base includes scores.

10. The method of claim 1, wherein one or more of the sheets are translucent or substantially transparent.

11. The method of claim 1, wherein the chemical reaction is for polymerase chain reaction.

12. An apparatus, comprising:
a first pliable sheet
a second pliable sheet coupled to the first pliable sheet to define a reaction chamber between the first pliable sheet and the second pliable sheet, the reaction chamber to hold a fluid for a chemical reaction; and
a plurality of walls disposed in the reaction chamber, each of the walls extending from the first pliable sheet to the second pliable sheet to divide the reaction chamber into a plurality of chambers.

13. The apparatus of claim 12, wherein at least one of the first pliable sheet and the second pliable sheet define a mixing chamber fluidly coupled to the reaction chamber.

14. The apparatus of claim 13, further comprising a removable fluid seal between the mixing chamber and the reaction chamber.

15. The apparatus of claim 12, wherein a base of the reaction chamber includes scores.

16. The apparatus of claim 12, further comprising a brace to impart rigidity to the reaction chamber.

17. The apparatus of claim 12, wherein the sheets are to be dispensed from one or more rolls of pliable material.

18. The apparatus of claim 12, wherein the reaction chamber has a thickness between about 50 microns and about 200 microns.

19. The apparatus of claim 12, wherein one or more of the sheets are translucent or substantially transparent.

20. The apparatus of claim 12, wherein the chemical reaction is for polymerase chain reaction.

21. A method, comprising:
coupling a first pliable sheet to a second pliable sheet to define a first chamber and a second chamber, the second chamber to be fluidly coupled to the first chamber, the first chamber having a thickness between about 50 microns and about 200 microns; and
dividing the first chamber into a plurality of chambers.

22. The method of claim 21, further comprising coupling a brace to the first chamber to impart rigidity to the first chamber.

23. The method of claim 21, wherein a base of the first chamber is substantially planar.

24. The method of claim 21, wherein the first chamber is to hold a fluid for polymerase chain reaction.

25. The method of claim 21, further comprising forming a removable fluid seal between the first chamber and the second chamber.

26. The method of claim 21, further comprising sealing the first chamber.

27. The method of claim 21, wherein a base of the first chamber includes scores.

28. The method of claim 21, wherein one or more of the sheets are translucent or substantially transparent.
